(19) United States
(12) Patent Application Publication
(10) Pub. No.: US 2017/0223481 A1
(43) Pub. Date: Aug. 3, 2017

(54) SKIN MOISTURE TESTING SYSTEM AND METHOD

(71) Applicant: Shenzhen Hali-Power Industrial Co., Ltd., Shenzhen (CN)

(72) Inventor: Xiaomin ZHU, Shenzhen (CN)

(21) Appl. No.: 15/159,768

(22) Filed: May 19, 2016

Related U.S. Application Data
(63) Continuation-in-part of application No. 15/014,041, filed on Feb. 3, 2016.

(51) Int. Cl. H04W 4/00; A61B 5/00; A61B 5/053; H04W 4/06

(52) U.S. Cl. CPC H04W 4/008; H04W 4/06; A61B 5/443; A61B 5/0531; A61B 5/7282; A61B 5/002

(57) ABSTRACT

A skin moisture testing system comprises a MCU control unit, an antenna impedance matching unit, a data preprocessing unit, a first contact, and a second contact. A skin moisture testing method matches with the MCU control unit to test a user's skin for a long time, and the tested skin moisture value can be visually displayed on an APP of a terminal. The tested skin moisture value can be made to form a graph.

### CROSS REFERENCE TO RELATED APPLICATIONS

This application is a continuation-in-part of U.S. patent application Ser. No. 15/014,041, entitled “SKIN MOISTURE TESTING SYSTEM AND METHOD” filed on Feb. 3, 2016.

### BACKGROUND

1. Technical Field

The present disclosure generally relates to a testing region, and especially relates to a skin moisture testing system and a method of testing skin moisture value.

2. Description of Related Art

Traditional cosmetics can only provide skin moisture; however, the traditional cosmetics do not know about skin moisture conditions, and cannot provide a scientific and reasonable moisturizing solution to the skin. The present invention aims to let consumers intuitively know his/her own skin condition, and provide the skin moisture in time. We can learn more about our skin, and care for our skin more scientifically and reasonably.

Therefore, a need exists in the industry to overcome the described problems.

### SUMMARY

The disclosure is to offer a skin moisture testing system and a method of testing the skin moisture value.

A skin moisture testing system comprises a MCU control unit, an antenna impedance matching unit, a data preprocessing unit, a first contact, a second contact, the first contact and the second contact in contact with skin of a user; the MCU control unit generating a first signal and transmitting the first signal to the first contact, the first signal transmitted to the second contact by a skin of a user and changed to a second signal, data preprocessing unit receiving the second signal from the second contact and filtering and shaping the second signal to a third signal, the MCU control unit sampling the third signal to get a plurality of sampling signals, the plurality of sampling signals quantified to get a semaphore value, and the semaphore value translated into a skin impedance value, then the skin impedance value translated into a skin moisture content value corresponding to the skin impedance value; the antenna impedance matching unit matching with the MCU control unit, and partly matching with an antenna of a terminal.

Preferably, the high frequency signal is a 4 KHz±500 Hz sinusoidal signal.

Preferably, a ninth terminal of the MCU control unit is connected with the Data preprocessing unit, and is used to detect integral values.

Preferably, a fifth terminal of the MCU control unit is connected with a resistor and a light emitting diode, and is used to control the light emitting diode.

Preferably, a fifteenth terminal of the MCU control unit emits the 4 KHz±500 Hz sinusoidal signal and transmits the 4 KHz±500 Hz sinusoidal signal to the first contact.

Preferably, a thirty first terminal of the MCU control unit and a thirty second terminal of the MCU control unit are connected with the Antenna impedance matching unit.

Preferably, a circuit of the antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the MCU control unit, another lead between the first coil and the second coil is connected with the thirty first terminal of the MCU control unit, a lead between the second coil and the third capacitor is connected with a thirtieth terminal of the MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

Preferably, the MCU control unit is a chip having 48 foot, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

A method of testing skin moisture value using a skin moisture system comprising a MCU control unit and two contacts, including: an application installed in a terminal having an antenna; the MCU control unit transmitting a broadcast; the terminal recognizing the broadcast after the antenna matching with the MCU control unit; the two contacts contacting a skin of a user and the skin moisture testing system testing the skin moisture value of the user; and a tested skin moisture value calculated by the MCU control unit and transmitted to the terminal to display to the user.

Preferably, the MCU control unit transmits the broadcast by pressing the power button.

Preferably, before the terminal Bluetooth antenna is matched and connected with the MCU control unit, the user selects to boot the terminal Bluetooth antenna on the terminal, then selects a corresponding service set identifier of the broadcast on the application of the terminal.

Preferably, the skin moisture testing system further comprises a Antenna impedance matching unit, a Data preprocessing unit, a signal transmitting module, a first contact, a second contact, the first contact and the second contact in contact with skin of the user.

Preferably, the first signal is a high frequency signal which is a 4 KHz±500 Hz sinusoidal signal.

Preferably, a ninth terminal of the MCU control unit is connected with the Data preprocessing unit, and is used to detect integral values.

Preferably, a fifth terminal of the MCU control unit is connected with a resistor and a light emitting diode, and is used to control the light emitting diode.

Preferably, a fifteenth terminal of the MCU control unit emits the 4 KHz±500 Hz sinusoidal signal and transmits the 4 KHz±500 Hz sinusoidal signal to the first contact.

Preferably, a thirty first terminal of the MCU control unit and a thirty second terminal of the MCU control unit are connected with the Antenna impedance matching unit.

Preferably, a circuit of the Antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the MCU control unit, another lead between the first coil and the second coil is connected with the thirty first terminal of the MCU control unit, a lead between the second coil and the third capacitor is connected with a thirtieth terminal of the MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

Preferably, the MCU control unit has a power button, and is used to do AD testing module, LED indication controlling module, antenna impedance matching, and the MCU control unit is a chip having 48 terminals, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

Compared to the traditional skin moisture testing system, the skin moisture testing method of the present invention is motivated by a pulse signal, the skin moisture testing method can effectively and accurately test skin moisture value. The skin moisture testing method also matches with a lower powered Bluetooth MCU to test user's skin for a long time, then the tested skin moisture value can be visually displayed on the APP of the terminal, the testing data can be made to form a graph, such that the skin moisture testing method can test comprehensive skin nutrition moisture percentage under the basis of an guarantee of no damaging to the skin, and the skin moisture testing method can guide the care of the skin.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is a schematic diagram of a skin moisture testing system;

FIG. 2 is an isometric view of a circuit structure;

FIG. 3 is an isometric view of a MCU control unit;

FIG. 4 is an isometric view of a Antenna impedance matching unit;

FIG. 5 is a structure diagram of a signal transmitting module;

FIG. 6 is a structure diagram of a data preprocessing unit;

FIG. 7 is a flow chart of a method of testing a skin moisture value based on the skin moisture testing system;

FIG. 8 is a schematic diagram of being sampled a plurality of sampling signals by the MCU control unit.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

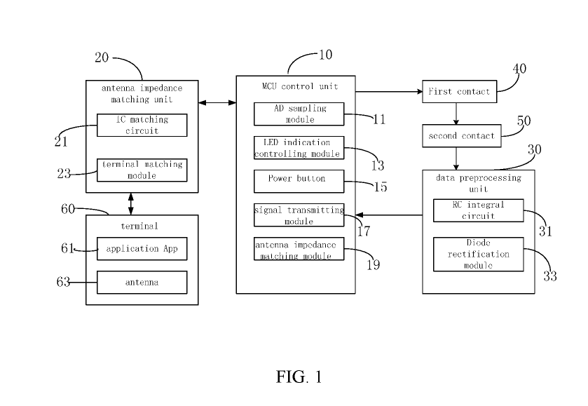

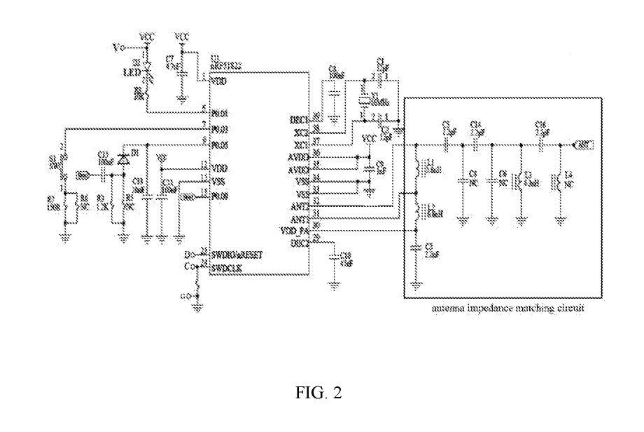

Referring to FIG. 1 and FIG. 2, a skin moisture testing system includes a MCU control unit 10 (Micro controller Unit, MCU), an antenna impedance matching unit 20, a data preprocessing unit 30 which includes a RC integral circuit 31 and a diode rectification module 33, a first contact 40 which is presented as Skin 1, a second contact 50 which is presented as Skin 2, wherein the first contact 40 and the second contact 50 are in contact with a skin of a user. The MCU control unit 10 generates a first signal and transmits the first signal to the first contact 40. The first contact 40 transmits the first signal to the second contact 50 by the skin, and the first signal is changed to a second signal when the first signal flow through the skin. The second contact 50 transmits the second signal to the data preprocessing unit 30. In the data preprocessing unit 30, the second signal is filtered and shaped, and is changed to a third signal. The MCU control unit 10 receives the third signal from the data preprocessing unit 30, and sampling the third signal to get a plurality of sampling signals. The plurality of sampling signals are quantified to get a semaphore value, and the semaphore value is translated into a skin impedance value. The skin impedance value is translated into a skin moisture content value corresponding to the skin impedance value by means of data fitting analysis method. The antenna impedance matching unit 20 receives the skin moisture content value from the MCU control unit 10, and transmits the skin moisture content value to a terminal 60.

In one embodiment, the first signal is a sinusoidal signal, especially is a square wave of high frequency sinusoidal signal. The second signal is similar to a sinusoidal signal. The third signal is a sinusoidal signal.

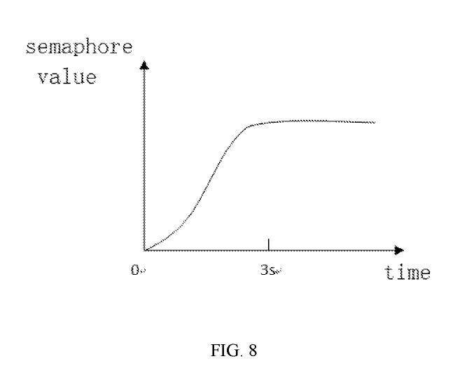

Referring to FIG. 8, in one embodiment, when the first contact 40 and the second contact 50 contact with the skin, during 0-3 seconds the plurality of sampling signals get by the MCU control unit 10 are in an instable state, subsequently the sampling signals will gradually increase until the difference between before and after sampling signals is less than 2 after 3 seconds, herein the plurality of sampling signals are in the stable state. And then, average of the sampling signals between 3 to 4 seconds is obtain, and the average of the sampling signals is translated into the skin moisture content value and is transmitted to the terminal 60.

Referring to FIG. 1 and FIG. 2, the MCU control unit 10 includes a AD sampling module 11, a LED indication controlling module 13 (light emitting diode, LED), a power button 15, a signal transmitting module 17, an antenna impedance matching module 19. The antenna impedance matching unit 20 includes an IC matching circuit 21 and a terminal matching circuit 23, the signal transmitting module 17 can transmit the first signal to the first contact 40, after a current passes through the skin, at this time the first signal has already been changed to the second signal, the second signal can be signal filtered and shaping treated by the data preprocessing unit 30, and is changed to the third signal. In the MCU control unit 10, the third signal is sampled to get a plurality of sampling signals, and then the sampling signals are quantified to get a semaphore value, then the semaphore value is translated into a skin impedance value through the data fitting analysis method, finally the skin impedance value is translate into a skin moisture content value corresponding to the skin impedance value. The antenna impedance matching unit 20 is responsible for matching with the antenna impedance matching module 19, and matching with an antenna 63 of the terminal 60, such that the skin moisture testing system of the present invention can remotely and reliably transport the skin moisture content value.

In one embodiment, the antenna impedance matching module 19, the antenna impedance matching unit 20, and the antenna 63 of the terminal 60 transmit signal to each other through Bluetooth.

The high frequency sinusoidal signal can be 4 KHz±500 Hz sinusoidal signal, such as 3700 Hz, 3800 Hz, 3900 Hz, 4000 Hz, 4300 Hz, and 4400 Hz.

A ninth terminal of the MCU control unit 10 is connected with the data preprocessing unit 30, and can be used to test integral values.

A fifth terminal of the MCU control unit 10 is connected with a resistor R8 and a light emitting diode (LED), and can be used to control a state of the light emitting diode.

A fifteenth terminal of the MCU control unit 10 emits 4 KHz±500 Hz sinusoidal signal and transmits the 4 KHz±500 Hz sinusoidal signal to the first contact 40.

A thirty first terminal of the MCU control unit 10 and a thirty second terminal of the MCU control unit 10 are connected with the antenna impedance matching unit 20.

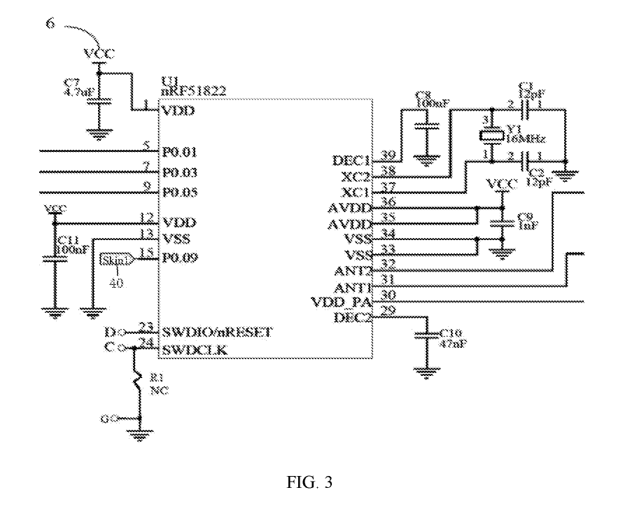

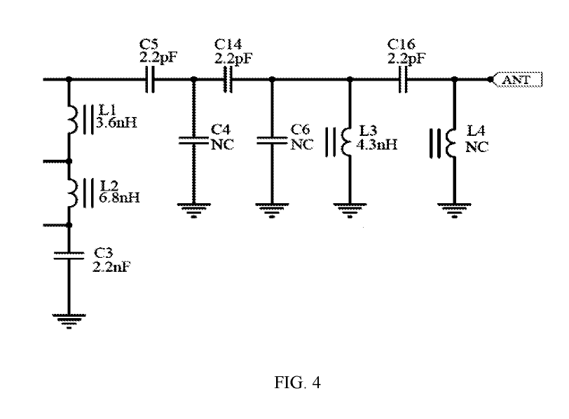

Referring to FIG. 3 and FIG. 4, a circuit of the antenna impedance matching unit 20 includes a first coil L1, a second coil L2, a third coil L3, a fourth coil L4, a third capacitor C3, a fourth capacitor C4, a fifth capacitor C5, a sixth capacitor C6, a fourteenth capacitor C14, a sixteenth capacitor C16, and an antenna; the antenna, the sixteenth capacitor C16, the fourteenth capacitor C14, the fifth capacitor C5, the first coil L1, the second coil L2, the third capacitor C3 are cascaded with each other in sequence, and then grounded, a lead between the first coil L1 and the fifth capacitor C5 is connected with the thirty second terminal of the MCU control unit 10, another lead between the first coil L1 and the second coil L2 is connected with the thirty first terminal of the MCU control unit 10, a lead between the second coil L2 and the third capacitor C3 is connected with a thirtieth terminal of the MCU control unit 10, the fourth capacitor C4 is connected with the fifth capacitor C5 and the fourteenth capacitor C14, and then grounded, the fourth capacitor C4 is located between the fifth capacitor C5 and the fourteenth capacitor C14, the fourteenth capacitor C14 and the sixteenth capacitor C16 are connected with the sixth capacitor C6 and the second coil L3 in parallel, and then grounded, the fourth coil L4 is connected with the sixteenth capacitor C16 and the antenna, and then grounded, the fourth coil L4 is located between the sixteenth capacitor C16 and the antenna.

In at least one exemplary embodiment, the MCU control unit 10 can be a chip which can have forty eight terminals, a first terminal of the chip is connected with a power VCC 6 (Volt Current Condenser, VCC), and the first terminal of the chip is grounded through a seventh capacitor C7; a seventh terminal of the chip is connected with a switch (not shown); a twelfth terminal of the chip is connected with the power VCC 6, and the twelfth terminal of the chip is grounded through a eleven capacitor C11; a thirteen terminal of the chip is grounded; a twenty fourth terminal is grounded by a first resistor R1, a twenty third terminal is connected with a D point, the twenty fourth terminal is connected with a C point, a G point is grounded, and the D point, the C point and the G point are burning points; a twenty ninth terminal is grounded through a tenth capacitor C10, a thirty third terminal and a thirty fourth terminal are both grounded, a thirty fifth terminal and a thirty sixth terminal are both connected with the power VCC 6, the thirty fifth terminal and the thirty sixth terminal are grounded through a ninth capacitor C9, a thirty seventh terminal is grounded through a first capacitor C1, a thirty eighth terminal is grounded through the second capacitor C2, a crystal oscillator Y1 is located between the thirty seventh terminal and the thirty eighth terminal, and the crystal oscillator Y1 is connected with the thirty seventh terminal and the thirty eighth terminal, a thirty ninth terminal is grounded through a eighth capacitor C8.

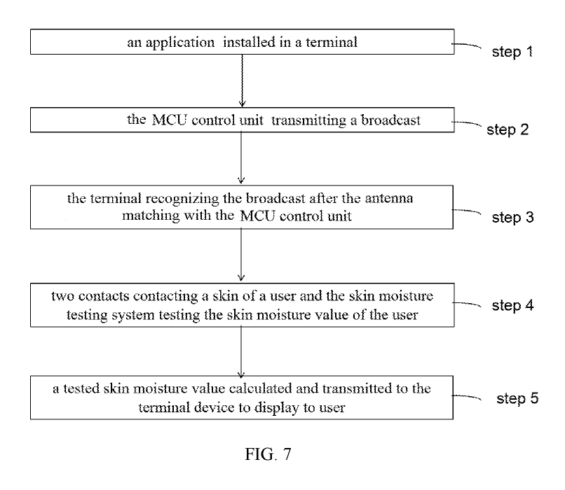

Referring to FIG. 7, FIG. 7 is a flow chart of a method of testing a skin moisture value based on the skin moisture testing system which includes the MCU control unit 10 and two contacts, wherein the two contacts includes the first contact 40 and the second contact 50. The method of testing the skin moisture value based on the skin moisture testing system, the method comprising: step 1: an application 61 installed in an terminal having an antenna 63, the terminal 60 being a mobile phone or a tablet personal computer; step 2: the MCU control unit 10 transmitting a broadcast by pressing the power button 15; step 3: the terminal 60 recognizing the broadcast after the terminal antenna 63 matching with the MCU control unit 10; step 4: the two contacts contacting a skin of a user and the skin moisture testing system testing the skin moisture value of the user; and step 5: a tested skin moisture value calculated by the MCU control unit 10 and transmitted to the terminal 60 to display to the user. Further, the tested skin moisture value can be made to form a graph. In one embodiment, the antenna 63 is a Bluetooth antenna.

It is to be understood that, when the MCU control unit 10 sends out the broadcast, the user can select to boot the antenna 63 on the terminal 60, then the user can select a corresponding service set identifier (SSID) of the broadcast on an APP interface of the terminal 60, after the terminal 60 are matched and connected with the skin moisture testing system, the first contact 40 and the second contact 50 can be contacted with the skin.

Referring to FIG. 1, FIG. 1 is a schematic diagram of the skin moisture testing system, the skin moisture testing system of the present invention includes the MCU control unit 10, the antenna impedance matching unit 20, the data preprocessing unit 30, a first contact 40, a second contact 50, and a terminal 60.

The skin moisture testing system can be connected with the terminal 60 and send data to the terminal 60 through the Bluetooth. The terminal 60 can be a phone, a tablet personal computer and so on. The skin moisture testing system can test percentage of comprehensive nutrient water in the skin based on an assurance of not damaging the skin, and a skin care can be guided by a skin moisture testing product using the skin moisture testing system. Testing parts of the skin can include head, face, eye socket, neck, and so on. Firstly, the application 61 can be installed on the terminal 60, and the application 61 can be a MiLi Pure application. The MCU control unit 10 transmits a broadcast, and the terminal 60 recognizes the broadcast after the antenna 63 matching with the MCU control unit 10. The first contact 40 and the second contact 50 contacts the skin of the user and the skin moisture testing system tests the skin moisture value of the user. A tested skin moisture value is calculated by the MCU control unit 10 and is transmitted to the terminal 60 to display to the user.

The square wave of the high frequency sinusoidal signal can be applied on the first contact 40 which is contacted with the skin, the high frequency sinusoidal signal can be 4 KHz±500 Hz sinusoidal signal, a weak small current passes through the skin and then return back, after the sinusoidal signal is signal filtered, and shaping treated, the MCU control unit 10 starts sampling to get a plurality of samples, the samples are quantified to get a semaphore value, the semaphore value can be transformed into a corresponding skin impedance value, a transformation equation can be established through the data fitting analysis method, finally the skin impedance value is translated into skin moisture content value corresponding to the skin impedance value. Tested skin moisture value can be calculated by the MCU control unit 10, and then be sent to the application 61 of the terminal 60, such that user can observe the value visually.

Referring to FIG. 3, FIG. 3 is an isometric view of the MCU control unit 10; the MCU control unit 10 is responsible for AD sampling testing, sending and receiving data signal, logic control signal, and sending high frequency sinusoidal signal. The ninth terminal is connected with a testing integral value. The fifth terminal is connected with the resistor R8 and the light emitting diode to control a state of the light emitting diode. The fifteenth terminal sends out 4 KHz sinusoidal signal to the first contract 40. The thirty first terminal and the thirty second terminal are connected with the antenna impedance matching unit 20.

Referring to FIG. 4, FIG. 4 is an isometric view of the antenna impedance matching unit 20, the Antenna impedance matching unit 20 is responsible for matching with the MCU control unit 10, and matching with the antenna 63, such that the skin moisture testing product using the skin moisture testing system can transmit the data to the terminal 60 reliably and remotely.

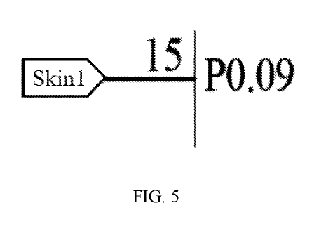

Referring to FIG. 5, FIG. 5 is a structure diagram of the signal transmitting module 17, the signal transmitting module 17 is responsible for transmitting 4 KHZ sinusoidal signal emitted by the MCU control unit 10 to the first contact 40.

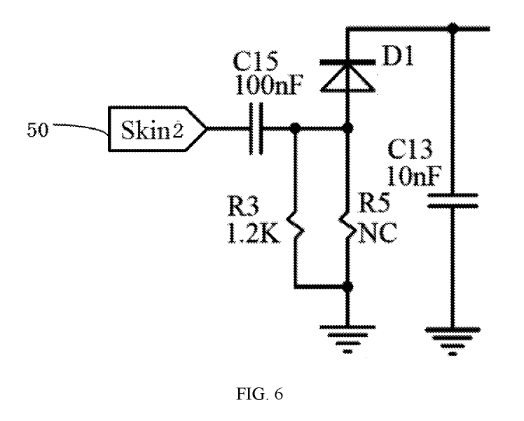

Referring to FIG. 6, FIG. 6 is a structure diagram of the data preprocessing unit 30, a sinusoidal signal emitted by the MCU control unit 10 can pass through the skin, and be transmitted to the data preprocessing unit 30 through the second contract 50 of the skin moisture testing product using the skin moisture testing system, at this time the sinusoidal signal has already changed, the changed sinusoidal signal is integrated, filtered and shaping treated through the RC circuit 31 which is formed by the fifteenth capacitor C15 and the resistor R3, then the changed sinusoidal signal is returned back to MCU control unit 10, and the MCU control unit 10 starts AD sampling to get a plurality of sampling signals, the sampling signals are quantified to get a semaphore value, the semaphore value is transformed into corresponding skin impedance value. Then the skin impedance value is sent to the terminal 60.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A skin moisture testing system, comprising,
a MCU control unit, an antenna impedance matching unit, a data preprocessing unit, a first contact, a second contact, the first contact and the second contact in contact with a skin of a user;
the MCU control unit generating a first signal and transmitting the first signal to the first contact, the first signal transmitted to the second contact by the skin and changed to a second signal, the data preprocessing unit receiving the second signal from the second contact and filtering and shaping the second signal to a third signal, the MCU control unit sampling the third signal to get a plurality of sampling signals, the plurality of sampling signals quantified to get a semaphore value, and the semaphore value translated into a skin impedance value, then the skin impedance value translated into a skin moisture content value corresponding to the skin impedance value;
the antenna impedance matching unit matching with the MCU control unit, and matching with an antenna of a terminal.

2. The skin moisture testing system of claim 1, wherein when the first contact and the second contact contact with the skin, during 0-3 seconds the plurality of sampling signals get by the MCU control unit are in an instable state.

3. The skin moisture testing system of claim 2, wherein when the difference between before and after sampling signals is less than 2, the plurality of sampling signals get by the MCU control unit are in a stable state.

4. The skin moisture testing system of claim 1, wherein a fifth terminal of the MCU control unit is connected with a resistor and a light emitting diode, and is used to control the light emitting diode.

5. The skin moisture testing system of claim 2, wherein the first signal is a high frequency signal which is a 4 KHz±500 Hz sinusoidal signal, a fifteenth terminal of the MCU control unit emits the 4 KHz±500 Hz sinusoidal signal and transmits the 4 KHz±500 Hz sinusoidal signal to the first contact.

6. The skin moisture testing system of claim 1, wherein a thirty first terminal of the MCU control unit and a thirty second terminal of the MCU control unit are connected with the antenna impedance matching unit.

7. The skin moisture testing system of claim 6, wherein a circuit of the antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the MCU control unit, another lead between the first coil and the second coil is connected with the thirty first terminal of the MCU control unit, a lead between the second coil and the third capacitor is connected with a thirtieth terminal of the MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

8. The skin moisture testing system of claim 1, wherein the MCU control unit is a chip having a plurality of foot, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

9. A method of testing skin moisture value using a skin moisture system comprising a MCU control unit and two contacts, including:
an application installed in a terminal having an antenna;
the MCU control unit transmitting a broadcast;
the terminal recognizing the broadcast after the antenna matching with the MCU control unit;
the two contacts contacting a skin of a user and the skin moisture testing system testing the skin moisture value of the user;
and a tested skin moisture value calculated by the MCU control unit and transmitted to the terminal to display to the user.

10. The method of claim 9, wherein before the antenna is matched and connected with the MCU control unit, the user selects to boot the antenna on the terminal, then selects a corresponding service set identifier of the broadcast on the application of the terminal.

11. The method of claim 9, wherein the skin moisture testing system further comprises a antenna impedance matching unit and a data preprocessing unit, wherein the two contacts comprises a first contact and a second contact, and the first contact and the second contact in contact with skin of the user.

12. The method of claim 11, wherein when the skin moisture testing system testing the skin moisture value of the user, the MCU control unit generating a first signal and transmitting the first signal to the first contact, the first signal transmitted to the second contact by the skin and changed to a second signal, the data preprocessing unit receiving the second signal from the second contact and filtering and shaping the second signal to a third signal, the MCU control unit sampling the third signal to get a plurality of sampling signals, the plurality of sampling signals quantified to get a semaphore value, and the semaphore value translated into a skin impedance value, then the skin impedance value translated into a skin moisture content value corresponding to the skin impedance value.

13. The method of claim 12, wherein when the first contact and the second contact contact with the skin, during 0-3 seconds the plurality of sampling signals get by the MCU control unit are in an instable state; when the difference between before and after sampling signals is less than 2, the plurality of sampling signals get by the MCU control unit are in a stable state.

14. The method of claim 12, wherein the first signal is a high frequency signal which is a 4 KHz±500 Hz sinusoidal signal, a fifteenth terminal of the MCU control unit emits the 4 KHz 500 Hz sinusoidal signal and transmits the 4 KHz±500 Hz sinusoidal signal to the first contact.

15. The method of claim 11, wherein a ninth terminal of the MCU control unit is connected with the data preprocessing unit, and is used to detect integral values.

16. The method of claim 11, wherein a fifth terminal of the MCU control unit is connected with a resistor and a light emitting diode, and is used to control the light emitting diode.

17. The method of claim 11, wherein a thirty first terminal of the MCU control unit and a thirty second terminal of the MCU control unit are connected with the antenna impedance matching unit.

18. The method of claim 17, wherein a circuit of the antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the MCU control unit, another lead between the first coil and the second coil is connected with the thirty first terminal of the MCU control unit, a lead between the second coil and the third capacitor is connected with a thirtieth terminal of the MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

19. The method of claim 11, wherein the MCU control unit has a power button, and is used to do AD testing module, LED indication controlling module, antenna impedance matching, and the MCU control unit is a chip having 48 terminals, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.
